(19) United States
(12) Patent Application Publication
(10) Pub. No.: US 2016/0038442 A1
(43) Pub. Date: Feb. 11, 2016

(54) MATERIALS AND METHODS FOR CONTROLLING INFECTIONS WITH NEGATIVE PRESSURE WOUND THERAPY

(71) Applicant: INNOVATION TECHNOLOGIES, INC., Lawrenceville, GA (US)

(72) Inventors: Carolyn L. TWOMEY, Lawrenceville, GA (US); Gareth CLARKE, Lawrenceville, GA (US); Samuel L. ZAIDSPINER, Lawrenceville, GA (US)

(21) Appl. No.: 14/815,966

(22) Filed: Aug. 1, 2015

Related U.S. Application Data
(60) Provisional application No. 62/034,913, filed on Aug. 8, 2014.

(51) Int. Cl. A61K 31/155; A61L 15/44

(52) U.S. Cl. CPC A61K 31/155; A61L 15/44; A61L 2300/404; A61L 2300/206

(57) ABSTRACT

The subject invention provides a method for promoting wound healing and preventing an infection or treating an existing infection with negative pressure wound therapy, said method comprising the steps of providing a sterile disinfectant composition comprising an active agent comprising chlorhexidine and administering the sterile disinfectant composition to the wound site prior to, during and/or following negative pressure wound therapy. The subject invention also provides a kit comprising the sterile disinfectant composition and, optionally, devices for administration of the sterile disinfection composition to the site with negative pressure wound therapy.

### CROSS-REFERENCE TO A RELATED APPLICATION

This application claims the priority benefit of U.S. Provisional Application Ser. No. 62/034,913, filed Aug. 8, 2014 which is incorporated herein by reference in its entirety.

### BACKGROUND OF THE INVENTION

The management and treatment of a wound, a surgical site, a surgical incision, or otherwise infection-prone tissues in the body, has three primary objectives: (1) prevention of infection, (2) preservation and/or restoration of function, and (3) preservation and/or restoration of cosmetic appearance. The most important of these objectives is the prevention of infection. Success in the prevention of infection directly affects the healing process and the degree to which function and cosmetic appearance can be preserved and/or restored.

Many different types of wound dressings are known for aiding in the healing process of a human or animal. These different types of wound dressings include many different types of materials and layers, for example, gauze, pads, foam pads or multi-layer wound dressings.

Negative pressure wound therapy (NPWT) is a therapeutic technique using a vacuum dressing to, for example, promote healing in acute or chronic wounds and to enhance healing of first and second degree burns. The therapy involves the controlled application of sub-atmospheric pressure to the local wound environment, using a sealed wound dressing connected to a vacuum pump. NPWT is widely recognized as a beneficial mechanism for improving the healing rate of a wound. Such therapy is applicable to a broad range of wounds such as incisional wounds, open wounds and abdominal wounds or the like.

NPWT therapy assists in the closure and healing of wounds by reducing tissue oedema; encouraging blood flow; stimulating the formation of granulation tissue; removing excess exudates and may reduce bacterial load and thus, infection to the wound. Furthermore, NPWT therapy permits less outside disturbance of the wound and promotes more rapid healing.

Devices for the negative pressure wound therapy are known in the art. For example, Kinetic Concepts, Inc. manufactures devices for NPWT (for example, the V.A.C.® device). U.S. Pat. Nos. 4,969,880; 5,100,396; 5,261,893; 5,527,293; 6,071,267; and 9,084,845 describe devices and methods to promote healing of a wound by the application of a negative pressure in the vicinity of the wound. These patents are incorporated herein by reference in their entirety. Commercially available devices for NPWT often use a wound dressing containing an open-cell polymer foam such as polyvinyl alcohol (PVA) or polyurethane (PU).

As with other wound therapies, one must also take into consideration the number and virulence of bacteria present at a wound site, which are critical determinants of whether the site becomes infected. Experimental evidence suggests that a critical level of bacteria is approximately 10⁵ organisms per gram of tissue. Below this level, a site or a tissue typically heals; at levels greater than 10⁵ bacteria per gram of tissue, infections often develop. Dirty wounds, or wounds that have not been treated within six hours, are likely to be contaminated with bacteria at levels that are higher than the critical level. Reducing the number of bacteria in and around the wound is critical for avoiding infection and expediting wound healing.

Chlorhexidine is a chemical antiseptic, used as an active ingredient in, for example, mouthwash designed to kill dental plaque and other oral bacteria. Chlorhexidine also has non-dental applications. For example, it is used for general skin cleansing, as a surgical scrub, and as a pre-operative skin preparation. Chlorhexidine is typically used in the form of acetate, gluconate, or hydrochloride, either alone or in combination with other antiseptics such as cetrimide.

### BRIEF SUMMARY OF THE INVENTION

The current invention provides materials and methods for improving negative pressure wound therapy (NPWT) and to overcome the disadvantages of the prior art. The invention provides materials and methods for promoting wound healing, including preventing or treating an infection of a wound by administering a sterile disinfectant composition comprising chlorhexidine, either directly or indirectly, to the wound that is undergoing NPWT.

Advantageously, it has been found that chlorhexidine-containing solutions can be administered to a subject according to the current invention without causing hemolysis or other deleterious effects on the blood, blood cells, or vascular system. Furthermore, when administered according to the procedures of the subject invention, the chlorhexidine- containing solutions of the subject invention do not result in deleterious absorption of chlorhexidine, systemic toxicity, or fibrosis.

Based on these findings it is now possible to utilize chlorhexidine-containing solutions in novel and advantageous ways, as described herein, to effectively treat and/or prevent infections and promote wound healing with NPWT.

Advantageously, the anti-microbial compositions of the subject invention are useful against drug resistant microbes, including MRSA. Furthermore, microbes do not readily acquire resistance to the treatments of the subject invention.

In a preferred embodiment, the active agent is chlorhexidine gluconate, preferably at a concentration of about 1.0% or less, more preferably at about 0.1% or less, and even more preferably at about 0.05% or less, and for some uses at 0.02% or less. Chlorhexidine dissolved in plain water or in a salt-containing solution, saline, for example, can be used according to the current invention.

In certain embodiments, the administration of the chlorhexidine-containing solution is followed by a rinse with, for example, saline. In other embodiments, no such rinse is applied.

The aqueous solution containing chlorhexidine may have other components including, for example, pH modifiers, buffers, local anesthetic agents, agents that promote wound healing (such as agents that help degrade biofilm) and other therapeutic and non-therapeutic components. In one embodiment, the composition to be applied to the wound “consists essentially” of an aqueous solution of CHG, which means that the solution contains no other active agent that materially changes the ability of the solution to control microbial growth.

The sterile disinfectant composition of the current invention can be used in a variety of applications directed at promoting wound healing and preventing and/or treating infections of wounds when utilizing NPWT. Treatment can be applied as a solution alone or in combination with a conventional wound or foam dressing used in NPWT.

The current invention also provides kits and trays comprising the sterile disinfectant composition and apparatuses or devices for administration of the sterile disinfectant composition to the subject. In preferred embodiments the kits and trays are sterile.

### DETAILED DESCRIPTION OF THE INVENTION

The current invention provides improved methods for promoting wound healing and preventing and/or reducing the development of an infection or treating an existing infection of a wound using negative pressure wound therapy (NPWT). In preferred embodiments, the NPWT utilizes pressure that is below atmospheric pressure.

Chlorhexidine-containing solutions can be administered to a subject according to the current invention without causing hemolysis or other deleterious effects on the blood, blood cells, or vascular system. Furthermore, when administered according to the procedures of the subject invention, the chlorhexidine-containing solutions of the subject invention do not result in deleterious absorption of chlorhexidine, systemic toxicity, or fibrosis. Furthermore, the compositions of the subject invention can be applied to tissue of the nervous system, including tissue of the central nervous system (CNS), without causing deleterious effects.

Based on these findings it is now possible to utilize chlorhexidine-containing solutions in novel and advantageous ways, as described herein, to effectively promote wound healing and to treat and/or prevent infections in wounds treated with NPWT.

Advantageously, the chlorhexidine solutions of the subject invention provide both a quick killing effect as well as a residual anti-microbial activity. Accordingly, soak times used with NPWT can be relatively short. The exact soak times can be determined by the skilled artisan having the benefit of the subject invention, in view of the characteristics of a particular wound. In certain embodiments, the soak times can be less than 5, 3, 2, or 1 minutes, or even less than a minute. Rinsing with saline is optional. In one embodiment, no saline rinse is used.

Advantageously, chlorhexidine has been found to bind to internal layers of the skin thereby providing residual anti-septic effect as well as preventing infections, thereby making the use of chlorhexidine in NPWT advantageous for wounds regardless of whether the wound is presently infected.

Advantageously, the anti-microbial compositions of the subject invention are useful against drug resistant microbes, including MRSA. Furthermore, microbes do not acquire resistance to the treatments of the subject invention.

In one embodiment, the method of the subject invention comprises the steps of:

(a) providing a sterile disinfectant composition comprising an active agent comprising, consisting essentially of, or consisting of, chlorhexidine at a concentration of about 1% or less, and

(b) administering the sterile disinfectant composition, directly or indirectly, to a wound before, during and/or following negative pressure wound therapy.

The administration of the chlorhexidine may be, for example, less than 10 minutes, 5 minutes, 4 minutes, 3 minutes, 2 minutes, or 1 minute, before, or after, the application of reduced pressure to the wound.

Preferably, the chlorhexidine is at a concentration of 0.05% or less. The term “wound” as used herein, may include burns, incisional wounds, excisional wounds, ulcers, abdominal wounds, traumatic wounds, and chronic open wounds that are treated with NPWT. The location of the wound can be any site that can be treated using NPWT.

The compositions of the current invention are combined with existing systems for NPWT. Exemplary NPWT that can benefit from the compositions of the subject invention include, but are not limited to, those described in U.S. Application Nos. 2011/0178481 and 2015/0209493; U.S. Pat. Nos. 4,969,880; 5,100,396; 5,261,893; 5,527,293; 6,071,267; 9,067,003; 9,084,845; and 9,089,630; and WO 1993/009727, each of which is hereby incorporated by reference in its entirety, including, for example, the cycling parameters set forth in U.S. Pat. No. 9,084,845.

The sterile disinfectant composition of the current invention preferably contains an active agent that preferably comprises chlorhexidine at a concentration of less than about 1%, less than about 0.1%, less than 0.75%, less than about 0.05%, less than about 0.025%, or less than about 0.02%. Typically the chlorhexidine solution would be greater than 0.005%, 0.01% or 0.02%. The chlorhexidine can be, for example, chlorhexidine gluconate (CHG), chlorhexidine acetate, chlorhexidine hydrochloride, or a combination thereof. The chlorhexidine may also be modified with, for example, a phosphate group to enhance efficacy, further reducing the likelihood of the development of resistant microbes. The sterile disinfectant composition can further contain one or more additional active agents. In certain embodiments, the composition contains no alcohol, or less than 5%, 10%, 25%, or 50% alcohol.

In certain embodiments, the disinfecting solution of the subject invention is incorporated into a wound dressing or foam such that the sterile disinfectant composition of the current invention is administered to the wound at a concentration of less than about 1%, less than about 0.1%, less than 0.75%, 0.05% or less, less than about 0.025%, or less than about 0.02%. Typically the chlorhexidine solution would reach the wound at 0.005%, 0.01% or 0.02%.

The foam may be, for example, hydrophilic, or a hydrophobic substance coated with a hydrophilic layer. The foam may be, for example, an open-cell foam. The dressing and/or foam may be adapted to have negative pressure applied and/or to remove exudates from the wound.

### Formulations and Spectrum of Activity

In one embodiment of the subject invention, a low concentration solution of chlorhexidine can be used to effectively prevent or treat infections. In specific embodiments, the chlorhexidine concentration is less than about 2%, less than about 1%, or less than about 0.1%. In a further embodiment, the chlorhexidine concentration is less than about 0.05%. In even further embodiments, the chlorhexidine concentration is between 0.02% and 0.075%. Specifically exemplified herein is the use of CHG.

In a specific embodiment, the CHG used according to the subject invention has the following chemical structure:

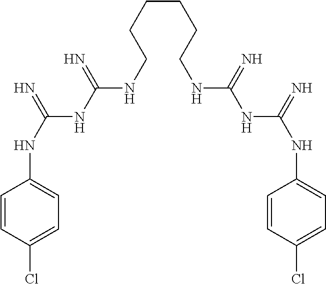 [SMILES: N=C(NCCCCCCNC(=N)NC(=N)Nc1ccc(Cl)cc1)NC(=N)Nc1ccc(Cl)cc1]

CHG

| | |
|---|---|
| Systematic | 1-[amino-[6-[amino-[amino-(4-chlorophenyl)amino- |
| (IUPAC) Name | methylidene]amino-methylidene]aminohexylimino] |
| | methyl]imino-N-(4-chlorophenyl)-methanediamine |

Chemical Data

| | |
|---|---|
| Formula | C₂₂H₃₀Cl₂N₁₀ |
| Mol. weight | 505.446 g/mol |

The pH of the sterile disinfectant composition is preferably neutral or slightly acidic. Preferably the pH is 5.0 to 7.5. More preferably the pH is 5.5 to 7.0.

In a preferred embodiment, the administration of the sterile disinfectant composition of the current invention to the wound results in a reduction in the number of bacteria or other microbes at the site when compared to either an untreated wound site or a wound site administered with saline or water that does not contain chlorhexidine. Advantageously, administration of the sterile disinfectant composition according to the subject invention can result in effective control of an infection and promote wound healing with negative pressure wound therapy without causing tissue damage.

Examples of additional active agents that can be administered to a subject in accordance with the subject invention include, but are not limited to, anti-bacterial agents, anti-viral agents, fungicidal agents, chemotherapeutic agents, topical antiseptics, anesthetic agents, oxygenated fluids and/or agents, antibiotics, diagnostic agents, homeopathic agents, and over-the-counter medications/agents. In one embodiment, the additional agent can be an anti-microbial peptide (AMP). AMPs are well known in the art.

In yet another embodiment, the additional agent is a diagnostic agent. The diagnostic agent may be, for example, an antibody, protein, or polynucleotide that binds to a target biomolecule. Any such binding may then be visualized utilizing technologies known to those skilled in the art.

Advantageously, the sterile disinfectant composition of the subject invention is effective in combating infection, even when organic materials (including blood, tissue, and/or dirt and debris) are present.

### Spectrum of Activity

Chlorhexidine is active against aerobic and anaerobic gram-positive and gram-negative bacteria. The drug also has some activity against Chlamydia trachomatis, certain fungi, and certain viruses.

Chlorhexidine is highly active against a variety of gram-positive aerobic bacteria, including Streptococcus mutants, S. pyogenes (group A β-hemolytic streptococci), S. salivarius, and S. sanguis. Chlorhexidine is active against Staphylococcus aureus, S. epidermidis, S. haemolyticus, S. hominis, and S. simulans. The drug is active against both oxacillin-resistant (ORSA) and oxacillin-susceptible staphylococci (also known as methicillin-resistant [MRSA] or methicillin-susceptible staphylococci).

Chlorhexidine is active against Enterococcus, including E. faecalis and E. faecium, and is active against both vancomycin-susceptible and vancomycin-resistant strains.

Chlorhexidine is also active against some anaerobic bacteria. The drug is active against some strains of Bacteroides, Propionibacterium, Clostridium difficile, and Selenomonas, but is less active against Veillonella.

### Fungi

Chlorhexidine has some activity against Candida albicans, C. dubliniensis, C. glabrata (formerly Torulopsis glabrata), C. guillermondii, C. kefyr (formerly C. pseudotropicalis), C. krusei, C. lusitaniae, and C. tropicalis (formerly C. parapsilosis). Chlorhexidine also has some activity against dermatophytes, including Epidermophyton floccosum, Microsporum gypseum, M. canis, and Trichophyton mentagrophytes.

Chlorhexidine also has antiviral activity against viruses that have a lipid component in their outer coat or have an outer envelope such as cytomegalovirus (CMV), human immunodeficiency virus (HIV), herpes simplex virus types 1 (HSV-1) and 2 (HSV-2), influenza virus, parainfluenza virus, and variola virus (smallpox virus).

In addition to killing bacteria, the sterile disinfectant composition of the subject invention can also “depathogenize” certain bacteria including, for example, Escherichia coli and Klebsiella aerogenes, making these bacteria less potent to cause infection.

The subject can be a mammal. Non-limiting examples of mammals that can be treated according to the methods of the current invention include humans, non-human primates, dogs, cats, equines, bovines, and pigs.

In one embodiment of the current invention, the sterile disinfectant composition is administered to a wound prior to, during and/or following NPWT.

The sterile disinfectant composition can be administered directly to the wound site as a plain aqueous solution, an isotonic solution, or other salt-containing solution of the active agent, prior to, during and/or following negative pressure wound therapy. In one embodiment, after a period of time sufficient for the active agent to kill and/or inhibit the growth of an infectious agent and promote wound healing, the wound site can be rinsed with a sterile solution free of the active agent. The period of time sufficient for the active agent to kill and/or inhibit the growth of the infectious agent can be about 1 minute to about 10 minutes, about 2 minutes to about 8 minutes, about 3 minutes to about 7 minutes, about 4 minutes to about 6 minutes, or about 5 minutes.

For the purpose of this invention, a plain aqueous solution of the active agent comprises the active agent and/or a second agent in a solution of water that is essentially devoid of solutes that provide osmolarity to the solution, for example, a salt or a sugar. For the purpose of this invention, an isotonic solution refers to a solution having the same osmotic pressure as blood. Typically, isotonic solutions contain about 0.85% of NaCl in water. Accordingly, an isotonic solution containing the active agent according to the current invention refers to a solution of the active agent and/or a second agent in about 0.85% NaCl in water.

In an even further embodiment of the current invention, the sterile disinfectant composition can be administered to a wound via a wound dressing or foam that is used with a negative pressure wound therapy device by incorporating the sterile disinfectant composition into or onto the wound dressing or foam.

For the purpose of this invention, a wound dressing or foam is manufactured for use with a NPWT device. In some cases the wound dressings or foams have an active agent incorporated therein, where the active agent is released over a period of time, including, for example, more than 5 minutes, more than 1 hour, more than 12 hours, more than one day, more than one week, more than one month, and/or more than one year.

Chlorhexidine is recognized to bind to healing tissues, for example, to skin, to provide antimicrobial and/or healing effect. Accordingly, the sterile disinfectant composition of the current invention provides an active agent that can bind to a healing tissue to enhance healing tissue recovery, prevent infection, and/or treat an existing infection, particularly when applied prior to, during and/or following negative pressure wound therapy. Where the solution is administered during and after negative pressure wound therapy, the solution can assist in reducing adhesion of the foam to the wound as well as removal of loose wound dressing/foam particles to reduce tissue traumatization when a wound dressing is removed and thus can assist in the healing process.

In one embodiment of the invention, the sterile disinfectant composition is left at the site after administration thereto. In a further embodiment of the invention, after administration of the sterile disinfectant composition of the current invention to a wound site or a tissue, the wound site or the tissue is rinsed with, for example, a sterile solution free of the active agent. Examples of sterile solutions free of the active agent include, but are not limited to, plain water and isotonic solutions free of the active agent. Rinsing with a solution free from the active agent ensures that the active agent is quickly and substantially removed from the site, thereby reducing harmful and unwanted side-effects exhibited by the active agent. The rinsing can be performed by administering the sterile solution free of the active agent to the site and removing the resultant solution from the site or the tissue by, for example, suction. In certain embodiments, the rinsing is performed within about 1 minute to about 10 minutes, about 2 minutes to about 5 minutes, or about 3 minutes from the time of administering the sterile disinfectant composition to the site in the subject.

A further embodiment of the current invention provides a kit comprising the sterile disinfectant composition and apparatuses or devices for administration of the sterile disinfectant composition to the site of the subject. The apparatuses and the devices for the administration of the sterile disinfectant composition to the site of the subject include, but are not limited to, a bottle or bag for administering the solution of chlorhexidine to the site to be used with negative pressure therapy.

Non-limiting examples of the kits and trays according to the current invention include an aqueous solution of the active agent, an isotonic solution of the active agent, a plain aqueous solution of the active agent at a 2× concentration of the active agent compared to the final working solution and a solution free of active agent having 2× isotonicity, the active agent in a solid form and sterile water or sterile isotonic solution, or a wound dressing or foam containing the active agent for use with a negative pressure wound therapy device.

The kits and trays (including custom packs) can be used to practice the methods of the current invention. For example, a user can use a kit comprising a solution of CHG by administering the solution of the active agent to the wound site of the subject prior to administering negative pressure wound therapy. Similarly, a user can mix equal amounts of the plain aqueous solution of the active agent at a 2× concentration and the solution free of active agent having 2× isotonicity to prepare a working isotonic solution of the active agent that can be used for topical wound solution instillation during negative pressure wound therapy. A user can also dissolve the active agent in the solid form in sterile water or sterile isotonic solution to prepare a working isotonic solution of the active agent.

Further, a user can administer the wound dressing or foam containing the active agent to the wound site of the subject with negative pressure wound therapy. Various types of wound dressings/foams and methods of making them are well known to a person of ordinary skill in the art and embodiments are within the purview of the current invention. For example, U.S. Application Publication Nos. 20110178481 and 20120046588, each of which is incorporated by reference in its entirety, describe such conventional wound dressings/foams that can be used in accordance with the subject invention.

The use of chlorhexide in wound irrigation applications has been previously described. See, for example, U.S. Published Application No. 2011-0288507A and U.S. Published Application No. 2011-0097372A, both of which are incorporated herein, by reference, in their entireties. Those patent applications describe various uses of CHG-containing solutions. In certain embodiments, the materials and compositions of the current invention specifically exclude those uses that were described in U.S. Published Patent Application Nos. 2011-0288507A and 2011-0097372A. The terms “about,” “approximately,” “approximate,” and “around” are used in this patent application to describe some quantitative aspects of the invention, for example, the concentration of the active agent. It should be understood that absolute accuracy is not required with respect to those aspects for the invention to operate. When these terms are used to describe a quantitative aspect of the invention the relevant aspect may be varied by up to ±10%. Thus, the terms “about,” “approximately,” “approximate,” and “around” allow for variation of the disclosed quantitative aspects of the invention by ±1%, ±2%, ±3%, ±4%, ±5%, ±6%, ±7%, ±8%, ±9%, or up to ±10%. For example, a sterile disinfectant composition comprising about 1% active agent can contain 0.9% to 1.1% active agent.

It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A method of treating a wound, comprising providing a sterile disinfectant composition comprising between about 0.005% and about 1.0% of chlorhexidine in an aqueous solution and administering the disinfectant composition to the wound, wherein the composition is administered to the wound before, during, and/or following negative pressure wound therapy.

2. The method according to claim 1, wherein the composition is administered to a wound at a concentration of 0.05% or less via a wound dressing or foam into which the composition is incorporated.

3. The method according to claim 2, wherein the composition reduces tissue traumatization when the dressing or foam is removed from the wound.

4. The method according to claim 1, further comprising rinsing the wound with sterile water or a sterile isotonic saline solution after the administration of the disinfectant composition.

5. The method according to claim 1, wherein the composition is administered to the wound by soaking the wound with the composition.

6. The method according to claim 1, wherein the wound is selected from burns, incisions, excisional wounds, ulcers, traumatic wounds, abdominal wounds, and chronic open wounds.

7. The method according to claim 1, which is effective in killing microbes and inhibiting residual microbial activity.

8. The method according to claim 7, wherein the microbe is a drug-resistant microbe.

9. The method, according to claim 1, wherein chlorhexidine contacts vasculature and/or nerves.

10. A wound dressing or foam, adapted for use in negative pressure wound therapy (NPWT), wherein said dressing or foam contains a composition that, upon use in NPWT, delivers to a wound chlorhexidine at a concentration of between about 0.005% and about 1.0%.

11. The wound dressing or foam according to claim 10, which is a foam.

12. A kit for treating a wound with negative pressure wound therapy, wherein said kit comprises a wound dressing or foam of claim 10.
